(19) United States
(12) Patent Application Publication
(10) Pub. No.: US 2007/0286935 A1
(43) Pub. Date: Dec. 13, 2007

(54) Bioactive Compositions, Natural Methods of Producing Them and Computational Methods for Designing Natural Production Processes

(75) Inventors: Martin Grigorov, Epalinges (CH); Peter Jan Van Bladeren, Chexbres (CH); Sunil Kochhar, Savigny (CH); Laurent-Bernard Fay, Evian (FR); Irene Corthesy-Theulaz, Epalinges (CH); Marcel-Alexandre Juillerat, Lausanne (CH); Michael Affolter, Savigny (CH); Ulrich Zachariae, Goettingen (DE)

Correspondence Address: BELL, BOYD & LLOYD LLP, P.O. Box 1135, CHICAGO, IL, 60690, US

(73) Assignee: NESTEC S.A., Vevey (CH)

(21) Appl. No.: 11/576,902

(22) PCT Filed: Oct. 7, 2005
(86) PCT No.: PCT/EP05/10837

§ 371 Date: Jul. 23, 2007

(30) Foreign Application Priority Data
Oct. 7, 2004 (US) 60617021

(51) Int. Cl. A23L 1/00; A23C 9/00; C07K 7/00; C12Q 1/02; C12Q 1/26; C12Q 1/34; G01N 33/02

(52) U.S. Cl. 426/580; 426/531; 426/594; 426/637; 435/12; 435/18; 435/29; 435/4; 436/20; 436/23; 530/326; 530/328; 530/330

(57) ABSTRACT

The present invention is directed to procedures for the controlled natural bioprocessing of naturally occurring biological molecules by processing activities present in select organisms, extracts of such organisms or other natural processing agents. The invention also covers the methods for developing these procedures and compositions prepared by the procedures.

### BACKGROUND OF THE INVENTION

The genomics-proteomics revolution of the 1990's has led to the identification and design of numerous small molecule drugs and potential drugs. In many cases these compounds do not occur in nature and many of them are toxic, antigenic, or have unsuitable pharmacokinetic properties.

Many bioactive molecules occur naturally in food stuffs as part of larger precursor molecules. Because they are components of foods that are commonly eaten, such agents have the potential to be less toxic to humans and animals. If methods could be found to release these compounds from their precursor food sources these molecules could be utilized to enrich foods or could be isolated and used as nutritional or therapeutic agents.

For example, PYY is a high affinity positive agonist of Y2 G-protein-coupled receptors (GPCR) and represents a relatively new class of therapeutic treatment for obesity, among other diseases. It is a natural hormone produced by specialized endocrine L-cells in the gut in proportion to the calorie content of a meal. PYY operates by reducing appetite and food intake by modulating appetite circuits in the hypothalamus. The agent has been shown to reduce caloric intake by 30% two hours after subjects, either obese or nonobese, received a 90-minute intravenous infusion. These subjects also experienced a significant decrease in their cumulative 24-hour caloric intake. In other studies, obese individuals have been observed to have lower levels of circulating PYY.

The present invention provides methods for the isolation of naturally occurring bioactive agents such as PYY and is directed to the resulting inventive compositions themselves. It also covers processes for developing these methods. These and other advantages of the present invention, as well as additional inventive features, will be apparent from the description of the invention provided herein.

### SUMMARY OF THE INVENTION

The present invention provides methods for producing functional foods enriched in functional biomolecules, procedures for developing those methods and the compositions prepared by those methods. The procedure first involves identifying the functional biomolecules to be generated. Attributes within these biomolecules that are necessary for function are then identified by analyzing the relationship between the biomolecule's structure and function. Precursor molecules containing this consensus motif, with all of the chemical and structural attributes required for activity, are then identified by electronically searching genomic databases of a variety of precursor food sources. Once a potential precursor molecule and food source is identified, another database search is carried out to identify organisms or enzymes that can be used to release from the precursor molecule a smaller compound containing all the attributes identified as necessary for function. The functional molecule can then be generated by treating the precursor with the processing agent(s) and releasing the functional biomolecule from its precursor. Functional molecules include bioactive molecules or biomaterials wherein the functions are bioactivity and self-organization and assembly, respectively. Precursor food sources include food stuffs or food grade proteins and their mixtures.

This approach provides a novel method for processing genome information in the design of combinations of raw nutritional sources and organisms which can liberate, in situ, active small molecules to enhance the nutritional and health-enhancing effect of the ingested functional food products.

To this end the invention provides a method for generating a bioactive molecule from a precursor food or protein source. The method includes the steps of identifying a bioactive molecule, identifying the attributes within the bioactive molecule that are necessary for bioactivity, identifying a precursor food source that contains a precursor molecule with the attributes necessary for bioactivity, identifying an agent that can be used to release from the precursor molecule a compound comprising the attributes necessary for bioactivity, and treating the precursor food source with the processing agent to thereby release from the precursor molecule a molecule that contains the attributes necessary for bioactivity.

In another aspect of the method the bioactive molecule can be a peptide, protein or nucleic acid polymer.

In another aspect, the method further comprises identifying chemical attributes within the bioactive molecule that are necessary for bioactivity.

In another aspect, the method further comprises identifying chemical attributes within a biomaterial that are necessary for self-organization/assembly into higher order structures.

In another aspect, the method further comprises identifying topological (two dimensional) and/or structural (three dimensional) attributes within the bioactive molecule that are necessary for bioactivity or self-organization/assembly.

In another aspect of the method the precursor food source can be derived from a plant such as rice, soy, maize, potato, coffee, milk, meat, and the like.

In another aspect, the method involves preparing a biomolecule using an agent that is a cell and the cell can be a lactobacteria, including Lactobacillus johnsonii (La1) or bifidobacteria, such as Bifidobacterium longum (B129) for example.

In another aspect, the method involves preparing a biomolecule using an agent that is an enzyme.

In another aspect, the method involves preparing a biomolecule using an agent that is an enzyme such as a protease, glycosidase, nuclease, oxidase or lipase or their combinations.

In another aspect of the invention the biomolecule binds to a receptor.

Certain embodiments of the invention are directed to a bioactive agent comprising a peptide generated from a food stuff or food grade protein comprising the amino acid sequence LNLV[TS][RK]X[RK][YFW], where X can be any naturally occurring amino acid and brackets denote the logical OR operation, and F, H, K, L, Q, R, W, Y are the standard amino acid abbreviations.

In certain embodiments of the invention the functional agent is isolated from a food stuff and the food stuff is rice.

In certain embodiments of the invention the food stuff is Oryza sativa.

In certain embodiments the bioactive agent is a peptide or protein that includes the sequence YSCRYFGYLVSKKKY (SEQ ID No. 1) derived from Arabidopsis thaliana amylogenin protein RGP or its closest analogue peptide sequences from the edible plant Oryza sativa, HSCRYFGYLVSRKKY (SEQ ID No. 2) found in the protein RGP2, or SACRCFGYMVSKKKY (SEQ ID No. 3) in the protein RGP1, or FDGVDFSEPLTRARF (SEQ ID No. 4) in the BiP protein.

In certain embodiments the bioactive agent is a peptide or protein that includes the sequence VWEKPWMDFK (SEQ ID No. 5), PWMDFK (SEQ ID No. 6), PWMDFKELQEFK (SEQ ID No. 7), PWMDF (SEQ ID No. 8), or VWEKPWMDF (SEQ ID No. 9) all derived from Oryza sativa oryzacystatin.

In certain embodiments of the invention the functional agent is generated by releasing it from a precursor molecule.

In certain embodiments of the invention the functional agent is generated by releasing it from a precursor molecule wherein the precursor molecule is in rice.

Additional features and advantages of the present invention are described in, and will be apparent from the following Detailed Description of the Invention.

### DETAILED DESCRIPTION OF THE INVENTION

For purposes of this specification the terms bioactive molecule and biomaterial are both considered to be functional biomolecules. The terms can be used interchangeably so long as their unique functions are kept in mind. Thus, methods and compositions described in this specification with respect to bioactive molecules are equally applicable to biomaterials.

Biomaterials are generally considered to be molecules that are capable of self-organization and assembly. Thus, biomaterials can be peptides that can form filaments and fibrils, hydrogels, surfactants and peptide hybrids when released from their precursor molecules.

The present invention provides methods for producing functional foods that are enriched in biomaterials or bioactive molecules such as bioactive peptides, proteins or nucleic acid polymers using natural food sources and food grade proteins. In addition, the present invention relates to the foods so designed.

In an embodiment, the present invention is directed to methods for developing natural bioprocessing procedures for producing enriched foods upon treatment with naturally occurring biological molecules, processing activities present in select organisms or extracts of such organisms or other natural processing agents. The invention also covers the various food compositions that can be prepared by the procedures, in addition to the bioactive molecules and methods for their production.

In the case of a bioactive molecule, the method generally involves identifying a bioactive molecule for production, identifying the attributes within the bioactive molecule that are necessary for bioactivity, identifying a precursor food source that contains a precursor molecule with those attributes, identifying an agent or agents that can be used to release from the precursor molecule a compound comprising the attributes necessary for bioactivity. Once a suitable processing method is identified, the method can be carried out by treating the precursor food source with the processing agent(s) to cause the release of a molecule that contains the attributes necessary for bioactivity.

The method also desirably includes steps for assessing the abundance of the potential precursor molecules. In addition, where the functional agent is derived from a precursor protein, the method also desirably includes an assessment of the stability of the potential precursor proteins towards peptidases and other enzymes or treatments that can be used to release it.

The functional agent can be any suitable biological molecule having a desired activity, especially peptides, proteins, nucleic acid polymers in addition to their derivatives which can be with lipids, saccharides, peptides and nucleic acids or their combinations. Functional agents can be excised from food stuffs, including food grade proteins, or other agricultural sources by the present methods. Generally, the desired bioactive molecules will specifically bind to a target receptor and activate or deactivate the receptor at concentrations that are obtainable from food processing. In certain embodiments where concentrations of the functional agent are lower than desired, chosen functional agents can be concentrated in the food source. Alternatively, extracts of the processed food stuff having elevated concentrations of functional agents can be prepared. Purification methods can also be used to purify agents partially or to substantially pure form. Methods for preparing concentrates, extracts, and for purification of functional agents necessarily vary depending on the nature of the target functional agent but these methods are well known in the art and can be easily carried out by one of skill in the art.

Precursor molecules can also be concentrated or purified and treated to release the bioactive molecules which can then be added back to food stuffs, as desired.

A variety of methods can be used to identify the attributes within the bioactive molecule that are necessary for bioactivity. For example, a literature search can be used and information can be gathered to determine how variations in the peptide, protein or nucleic acid polymer structure affect the desired activity and to identify all conserved amino acids, or other structures such as carbohydrate structures, nucleotides, or lipids that exist in the peptide or protein active agent. In addition, variations can often be generated by mutagenesis and the activity of the resulting compounds determined. Ultimately, a consensus motif that is responsible for activity can be obtained. The consensus motif will desirably contain all of the attributes within the bioactive molecule that are necessary for bioactivity and defines the simplest molecular framework that is known to be active. The molecular framework can include the chemical attributes within the molecule that are required for activity and structural attributes such as primary, secondary and tertiary structural requirements, in addition to modifications, such as glycosylation, acylation and the like.

A precursor food source that contains a molecule having all the attributes necessary for bioactivity can then be identified. Such food sources can be identified by searching for the occurrence of the consensus motif at both sequence and structural levels in large collections of genomic information including for example, ENSEMBL®, Nestlé genomic databases, and public genomic sequencing projects on food raw materials such as rice, soy, maize, potato, coffee and bovine milk. Precursor molecules found within these food sources can be proteins, peptides or nucleic acid polymers and their derivatives, depending on the nature of the desired biomolecule. Preferably when the agent is a peptide, such a search is performed using BLAST by searching for short nearly exact matches and/or by EMBOSS pattern matching module “patmatdb” by searching for short nearly exact matches. Where three dimensional structural motifs are required these structures can be found using structure prediction software, as is known in the art.

It is envisioned that a number of potential precursor molecules may be identified by this method. In such cases, it can be desirable to determine the abundance of the precursor molecules in order to identify precursors that will yield suitable amounts of the desired agent.

Alternatively, food grade proteins containing the consensus motif can be obtained and used in the preparation of the biomolecule.

A processing agent or agents can then be identified that can be used to release a compound from the precursor molecule such that the released compound contains all of the attributes necessary for bioactivity. The selected agents will, of course, depend on the nature of the functional agent to be released and the characteristics of the matrix in which it is found. Potential agents include microorganisms, extracts of microorganisms, proteolytic, glycolytic, nucleolytic, and lipolytic enzymes, oxidases, glycosidases, and chemical agents. In some instances these activities can be found in the genome repertoire of cells, such as probiotic bacteria.

Where proteases are required, the automated service offered by ExPASy website termed PeptideCutter, the module DIGEST from the bioinformatics software package EMBOSS, and/or the MEROPS protease database can be used to identify suitable enzyme activities. As is known, the PeptideCutter knowledge-based algorithm can be used to identify cleavage sites produced by a panel of more than 20 different proteases on protein sequences. (Keil, B., Specificity of Proteolysis, Springer-Verlag Berlin-Heidelberg-New York, 1992)

Certain methods utilize microorganisms to release the functional agent from its matrix. To identify microorganisms containing the desired activities, such as protease activities, the sequences of the enzymes thought to be useful can be compared to known bacterial genomes to identify similar protease sequences in those genomes. For example, the Bifidobacterium longum (B129) genome contains 74 protein sequences annotated as proteases or peptidases. One of them is highly similar to an Arg-C proteinase for which cleavage outcomes are computationally predictable by the PeptideCutter model. Thus, where an Arg-C proteinase activity is identified as a potential activity for use in releasing the bioactive agent from its source, the bacterium Bifidobacterium longum (B129), which contains that sequence, could be used. The Bifidobacteriun longum (B129) genome also has a sequence highly similar to sedolisin. It can be used in situations where both activities would be useful in releasing the bioactive agent from its precursor food source. This method is equally applicable with other enzyme activities.

Alternatively, the activities of probiotic bacterial strains can be identified and utilized without specific knowledge of the nature of the enzymes involved. At least with respect to proteases both intracellular and extracellular activity profiles have been reported. In a similar manner glycolytic, nucleolytic, and lipolytic activities can also be screened and the entire NCC bacterial collection evaluated to select the most promising strains.

Once both a precursor molecule is identified within a food source and processing agents are identified for releasing the functional agent, the method can be carried out by treating the food source with the processing agent(s) to release from the precursor molecules, molecules that contain the consensus motif. Preferably, the food source is treated with agents that are sufficient to release the functional agent in a single fermentation or treatment. However, when multiple processing agents are required and they are incompatible or require distinct environments for use, multiple processing steps can be undertaken.

The functional agent can be derived from a protein or peptide sequence. In such cases, precursor molecules can be identified for example by retrieving all known plant expressed sequence tags and proteins that contain the sequence of the bioactive agent. Variable or noncritical amino acids and critical amino acids in the sequence can be identified by analysis of published structure-activity relationships and by aligning the sequences of all known similar sequences. With this consensus motif a database containing all known plant proteins can be searched to identify the consensus motif within larger precursor proteins. A computational assessment of the tertiary structure can also be used to identify internal sequences in the identified precursors that will adopt a structure that is required for activity of the bioactive agent. This can be done using standard methods well known in the art. For example, where peptide sequences closely resemble known three dimensional structures, homology-modeling can be used. In cases where peptide sequences are more distant to known structures, fold-recognition protocols can be used. Where three-dimensional structure information of the receptor for the bioactive agent is available, the identified sequences can be analyzed by a variety of known modeling methods to determine whether their predicted structures are likely to bind to the receptor. The list of precursor proteins is then evaluated. An analysis can then be done by known methods, such as microarrays for genes differentially expressed in seeds and crops, to determine if the precursor molecule is sufficiently abundant to enable the preparation of biologically relevant amounts of the functional agent from the precursor. In addition, the sensitivity of the precursor molecule to proteases can be determined in order to evaluate the potential for releasing the target bioactive agent. Peptide synthesis of identified peptide sequences can be carried out and the peptides can then be tested for activity.

In one exemplary embodiment the generation of analogues to the bioactive peptide PYY3-36 can be selected. This compound is a ligand of the GPCR peptide hormone receptor Y, existing as subtypes Y1, Y2, Y4, and Y5. The receptor is involved in the regulation of satiety, the feeling of hunger. The PYY3-36 peptide activates its target receptors Y1 and Y2 at concentrations of about 0.5 nM. The method would also be useful in the identification and generation of other regulatory peptides against diseases such as diabetes and obesity, such as cholecystokinin (CCK), human growth hormone (HGH), and melanocortin, for example.

By way of example and not limitation, examples of the present invention can now be set forth.

### EXAMPLE 1

This example demonstrates the preparation of a bioactive agent having a positive agonist activity similar to PYY3-36. PYY3-36 is a ligand of the GPCR peptide hormone receptor Y, existing as subtypes Y1, Y2, Y4, and Y5. The receptor is involved in the regulation of satiety, the feeling of hunger, and blood pressure. It activates its target receptors Y1 and Y2 at concentrations of about 0.5 nM.

### Definition of the Simplest Bioactive Molecular Framework

An extensive literature search was conducted and was used to determine that only a small sequence in the C-terminal part PYY3-36 peptide is essential for appetite regulation and for binding to its cognate receptor. Variations around this sequence segment have been published. Several published sequences are gathered and shown below in Table 1. The amino acids in bold in Table 1 are highly conserved amino acids that constitute the simplest bioactive framework for PYY3-36.

NMR structure determination techniques have shown that the PYY3-36 peptide adopts a very particular three-dimensional shape known as the PP-turn type or fold. The N-terminal region is unstructured, while the C-terminal region forms a characteristic a-helical structure. Using this information, peptides having similar sequences and tertiary structures were identified to find potential homologues to the natural PYY3-36 ligand peptide.

The search was performed using EMBOSS “patmatdb” with the following pattern reproducing at best the chemical nature of the C-terminal fragment of the PYY3-36—XXX[RK]X[YFW]XXXX[TS][RK]X[RK][YFW]. Interestingly, tens of matching fragments occurring in various plant genomes were identified, several examples of which are shown in Table 1. The existence of the required α-helix structures of the target fragments was confirmed using peptide structure prediction software.

TABLE 1

| | |
|---|---|
| Target | SEQ ID No. |

| | | |
|---|---|---|
| Human peptide PYY3-36 | YPIKPEAPGEDASPEELNRYYASLRHYLNLVTRQRY | |
| Active epitope | ASLRHYLNLVTRQRY | |
| Published sequences | | |
| Sequence 11, US5604203 | ASLRHYLNLVARQRY | |
| Sequence 4, US56042703 | SLRHFLNLVTRQRY | |
| Sequence 10, US6075009 | FINLITRQRF | |
| Sequence 7, US5604203 | SLRHFLNLVTRQRY | |
| Sequence 13, US5604203 | ASLRHYENLVARQRY | |
| Sequence in A. thaliana genome | | |
| AtRGP, gi15237362, aa 81-95 | YSCRYFGYLVSKKKY | |
| Sequence in O. sativa genome | | |
| OsBIP, gi50904765, aa 317-331 | FDGTDFSEPLTRARF | |
| OsRGP1, gi34915 190, aa 93-105 | SACRCFGYMVSKKKY | |
| In vitro Tested Bioactive Peptides | | |
| Positive control | ASLRHYLNLVTRQRY | |
| AtRGP2 peptide fragment | YSCRYFGYLVSKKKY | 1 |
| OsRGP1 peptide fragment | SACRCFGYMVSKKKY | 2 |
| OsBIP peptide fragment | FDGVDFSEPLTRARF | 3 |

### Identified Hits For PY22-36 Analogues

Table 1 also identifies three potential bioactive peptides resulting from a search of known ESTs using the EMBOSS program. These sequences include two peptides from the protein Amylogenin, one from Arabidopsis thaliana (AtRGP2), the other from Oryza sativa (OsRGP1). The other potential peptide sequence was from a binding protein found in Oryza sativa (OsBiP).

Amylogenin is thought to be responsible for starch biosynthesis in plants and is also known as reversibly glycosylated protein (RGP). Analysis of Arabidopsis thaliana seed microarrays and microbiological data suggest that it is an abundant protein in seeds and roots, and that it is almost exclusively localized in plant Golgi membranes. BiP or binding protein is responsible for enhancing crop tolerance to environmental stress. Microbiological data suggest that BiP synthesis is coordinated with the onset of active storage protein in crops.

The peptides derived from the proteins AtRGP2, OsRGP1 and OsBiP were synthesized in 5 mg quantities at and purified to a purity of above about 90%. The peptides were tested in a competitive binding assay against PYY²²⁻³⁶ for binding to the GPCR receptor.

### Identifying Target Bioactive Molecular Frameworks. Search For Adequate Proteolytic Enzymes In the Genome Repertoire of Nestle Probiotic Bacteria

The automated free service offered by ExPASy website termed “PeptideCutter” and the module Digest from the bioinformatics software package EMBOSS was used to identify proteases that could release the PYY³⁻³⁶ analogue. Arg-C proteinase and sedolisin were identified as having activities that could release the bioactive agent from its native matrix. The sequences of the proteases used by PeptideCutter, MEROPS and Digest were compared to several bacterial genomes to check for the occurrence of highly similar sequences. For instance, in the Bifidobacterium longum genome 74 protein sequences are annotated as proteases or peptidases. Only one of them was found similar to the Arg-C proteinase for which cleavage outcomes are computationally predictable by the PeptideCutter model. Arg-C proteinase activity alone was not capable of producing the required active peptide. However, in the Bifidobacterium longum genome a sequence similar to sedolisin was identified. The combined activity of these two proteases in Bifidobacterium longum was shown to be capable of processing the Oryza sativa precursor protein into the desired PYY homolog.

### In Vitro/In Vivo Tests For Bioactivity

In-vitro receptor-based screening of receptor subtypes Y1, Y2, and Y3 was performed according to protocols known in the art. (Munoz et al., Mol. Cell. Endocrinol. 107, 77 (1995), Fuhlendorf et al., PNAS 87, 182 (1990)) Screening of OsBiP, ZmRGP1 and AtRGP1 was conducted on Y2-GPCR as described above using known methods and activity was measured as the percentage of inhibition of binding of the endogenous ligand PYY²²⁻³⁶ sequence. The PYY²²⁻³⁶ sequence was used as a positive control. Of the peptides tested, the one derived from AtRGP1 protein was able to inhibit binding of PYY²²⁻³⁶ by 20-40% when 10 μM concentration of both the PYY²²⁻³⁶ and OsBiP peptide were used in the binding assay. It is expected that the close analogue of the peptide derived from the Arabidopsis Thaliana protein AtRGP1 found in the rice protein OsRGP2, differing by only one chemically equivalent amino acid, should have similar activity. Using this technique OsBIP protein was also shown to bind the Y2 receptor the Y2 receptor. In vivo tissue-based screening on cultures of rat colon cells can be carried out according to known protocols, such as described in Dumont et al., Eur. J. Pharmacol. 238, 37 (1993).

### EXAMPLE 2

This example demonstrates the preparation of a bioactive agent that inhibits the effect of peptide CCK-4 activator on the CCK-B subtype receptor. CCK-8 and CCK-4 peptides are ligands of the CTPCR peptide hormone receptor CCK, which exists in at least two subtypes, A and B. CCK receptor subtype A is thought to be expressed in the gut and to be involved in the regulation of satiety, and the feeling of hunger. CCK receptor subtype B, also known as gastrin receptor, is thought to be involved in the secretion of gastric acid, and in the development of pathological conditions, such as of gastric ulcer and cancer. Antagonists of the CCK-B receptor can be used to treat these conditions.

### Definition of the Simplest Bioactive Molecular Framework

A common strategy in the design of antagonists is to mimic a positive agonist and to introduce molecular structure changes that enhance binding to the cognate receptor such that the antagonist occupies the binding site thereby preventing agonist binding. An extensive literature search and sequence comparison was used to determine that only a small sequence in the C-terminal part of human CCK peptides is essential for receptor binding. A summary of this information is provided in Table 2.

The amino acids in bold in Table 2 are highly conserved amino acids that constitute the simplest bioactive framework for CCK peptides.

The search was performed using EMBOSS “patmatdb” with the following pattern representing the chemical nature of the C-terminal fragment of CCK-peptides, —P[YFW]X[DE][YFW]. Tens of matching fragments occurring in various plant genomes were identified, several examples of which are shown in Table 2.

TABLE 2

| | |
|---|---|
| Target | SEQ ID No. |

| | | |
|---|---|---|
| Human peptide CCK-22 | NLQNLDPSHRISDRDYMGWMDF | |
| Human peptide CCK-8 activating | DYMGWMDF | |
| human CCK-A receptor | | |
| Human peptide CCK-4 activating | WMDF | |
| human CCK-B receptor | | |
| In vitro Tested Bioactive Peptides | | |
| Radiolabelled CCK-8 | DYMGWMDF | |
| Oryza sativa oryzacystatin hydrolyzed | VWEKPWMDFK | 5 |
| with trypsin − synthetic peptide 1 | | |
| Oryza sativa oryzacystatin hyrolyzed | PWMDFK | 6 |
| with trypsin − synthetic peptide 2 | | |
| Oryza sativa oryzacystatin hyrolyzed | PWMDFKELQEFK | 7 |
| with trypsin − synthetic peptide 3 | | |
| Oryza sativa oryzacystatin hyrolyzed | PWMDF | 8 |
| with trypsin + carboxypeptidase | VWEKPWMDF | 9 |

Table 2 also identifies two potential bioactive peptides from a search of known ESTs using the EMBOSS program. These two sequences include two fragment peptides from the protein oryzacystatin, from the organism Oryza sativa.

Oryzacystatin is a cystein proteinase inhibitor protein. Analysis of literature demonstrated that this protein is produced in high quantities in rice crops, that could reach 1 mg of protein per kilogram of crops. The peptides VWEKPWMDFK, PWMDFK and PWMDFKELQEFK were synthesized in 5 mg quantities and purified to a purity of above about 90%. The peptides were tested in a competitive binding assay against CCK-8 for binding to the CCK-B GPCR receptor.

### Identifying Target Bioactive Molecular Frameworks. Search For Adequate Proteolytic Enzymes

Commercial proteolytic enzymes such as trypsin or carboxypeptidase have been used to generate the active fragments from Oryza sativa rice.

### In Vitro Tests For Bioactivity

In-vitro receptor-based screening of receptor subtypes CCK-A and CCK-B was performed according to protocols known in the art. The binding activity of peptides VWEKPWMDFK, PWMDFK and PWMDFKELQEFK on CCK-A and CCK-B GPCR was measured as the percentage of inhibition of binding of the endogenous ligand CCK-8 sequence. The CCK-8 sequence was used as a positive control in this assay. Results showed that peptides VWEKPWMDFK, PWMDFK and PWMDFKELQEFK inhibited binding of the ligand CCK-8 to CCK-B receptor by 27%, 16%, and 5%, respectively at 10 μM concentration. None of the peptides tested activated CCK-A receptor at concentrations as high as 10 μM, probably due to the lack of sulfonation, a chemical modification required for agonizing the CCK-A receptor.

Hydrolysates were generated from the recombinantly expressed and purified oryzacystatin protein which was isolated from Oryza sativa. Trypsin was used to hydrolyze 10 mg of each sample using standard conditions. Two peptides were identified and purified from these hydrolysates, namely the VWEKPWMDFK and PWMDFK peptides, as these exhibited the highest activities in the first screen. Again the hydrolysate and the two purified peptides were submitted to receptor-based screening. The hydrolysate itself inhibited CCK-8 binding to the CCK-B receptor by 21% at a 100 μM hydrolysate concentration, while the purified peptides, VWEKPWMDFK and PWMDFK, inhibited CCK-8 binding by 16%, and 14% at a 10 μM concentrations.

A final optimization of the hydrolysis by the combined consecutive action of trypsin and limited carboxypeptidase digestion was carried out with the goal of obtaining the peptides VWEKPWMDF and PWMDF, resulting from the removal of the C-terminal lysine residue. The hydrolysate enriched in these peptides exhibited an increased inhibition of the binding of the ligand CCK-8 to the CCK-B receptor that reached 38% at a 10 μM concentration of the active peptides, in comparison to the 21% at the 10 times higher 100 μM concentration without trypsin treatment.

Trypsin digests were carried out by adding 96 mg urea to 200 μl oryzacystatin protein (2 mg) and incubating at room temperature until the urea dissolves (final concentration: ca. 4 M urea). To the urea solution 900 μl of 100mM ammonium bicarbonate in 1mM CaCl₂ is added followed by 120 μl of acetonitrile. 40 μg of trypsin (40 μg Promega sequencing grade trypsin in Promega resuspension buffer) was added and the solution incubated overnight at 37° C. A second aliquot of 10 μg trypsin was added followed by incubation for 3 h at 37° C.

50% of the digestion solution of oryzacystatin is stored at −20° C. for desalting by solid phase extraction using C18 reverse phase material and 50% of the digestion solution of oryzacystatin is subjected to carboxypeptiodase digestion.

Carboxypeptidase Y digestion was carried out on 720 μl (ca. 1 mg) of the tryptic digestion solution of oryzacystatin to which 1080 μl of 200 mM acetate buffer, pH 5 in 10% acetonitrile was added. 100 μg carboxypeptidase Y (Sigma Aldrich, carboxypeptidase Y solution was prepared three days before. It is good for one week according to Sigma Aldrich) is added and the solution incubated for 30 min at room temperature followed by the addition of 910 μl of 1% formic acid

A small fraction of each sample was characterized by nano LC-ESI-MSMS.

Both samples were desalted using 500 μl C18 solid extraction columns with 0.1% formic acid as washing solution and 80% acetonitrile/0.1% formic acid as elution solution.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A process for developing a method for generating functional biomolecules from precursors comprising:
identifying a functional biomolecule to be generated,
identifying attributes within the functional biomolecule that are necessary for function,
identifying a precursor food source that comprises a precursor molecule with the attributes necessary for function,
identifying an agent that can be used to release from the precursor a molecule that has the attributes necessary for function.

2. The method of claim 1, wherein the functional molecule is a bioactive molecule.

3. The method of claim 1, wherein the functional molecule is a biomaterial capable of self-organization/assembly.

4. The method of claim 1, wherein the functional molecule is selected from the group consisting of peptides, proteins, nucleic acid polymers and their combinations.

5. The method of claim 1, comprising identifying chemical attributes within the functional molecule that are necessary for function.

6. The method of claim 1, comprising identifying structural attributes within the functional molecule that are necessary for function.

7. The method of claim 1, wherein the precursor food source is a food stuff.

8. The method of claim 1, wherein the precursor food source is an enriched protein source.

9. The method of claim 1, wherein the precursor food source is selected from the group consisting of rice, soy, maize, potato, coffee and milk.

10. The method of claim 1, wherein the agent is a cell.

11. The method of claim 1, wherein the agent is a cell selected from the group consisting of lactobacteria and bifidobacteria strains.

12. The method of claim 1, wherein the agent is an enzyme.

13. The method of claim 1 wherein the agent is an enzyme selected from the group consisting of proteases, glycosidases, nucleases, oxidases, lipases and their combinations.

14. The method of claim 1, wherein the agent is a protease.

15. The method of claim 1, wherein the functional molecule is a bioactive molecule that binds to a receptor.

16. A method for preparing a functional food enriched in a functional biomolecule comprising:
identifying a functional biomolecule to be generated,
obtaining a precursor food source that contains a precursor molecule with the attributes necessary for function,
obtaining an agent that can be used to release from the precursor molecule a compound comprising the attributes necessary for function, and
treating the precursor food source with the agent to release from the precursor molecule the functional biomolecule.

17. The method of claim 16, wherein the functional biomolecule is a bioactive molecule.

18. The method of claim 16, wherein the functional biomolecule is a biomaterial capable of self-organization/assembly.

19. The method of claim 16, wherein the bioactive molecule is selected from the group consisting of peptides, proteins, nucleic acid polymers and their combinations.

20. The method of claim 16, wherein the precursor food source is selected from the group consisting of rice, soy, maize, potato, coffee and milk.

21. The method of claim 16, wherein the precursor food source is a food grade protein.

22. The method of claim 16, wherein the agent is a cell.

23. The method of claim 16, wherein the agent is an enzyme.

24. The method of claim 16, wherein the agent is an enzyme selected from the group consisting of proteases, glycosidases, nucleases, oxidases, and lipases and their combinations.

25. The method of claim 16, wherein the agent is a protease.

26. A bioactive agent comprising a peptide having an amino acid sequence of SEQ ID No. 8.

27. The bioactive agent of claim 26, wherein the peptide is in a food stuff.

28. The bioactive agent of claim 27, wherein the food stuff is Oryza sativa.

29. The bioactive agent of claim 26, wherein the peptide sequence comprises SEQ ID NO 9.

30. The bioactive agent of claim 26 wherein the peptide is generated by release from a precursor molecule.

31. The bioactive agent of claim 26 wherein the peptide is generated by release from a precursor molecule in rice.

32. A bioactive agent comprising a peptide comprising a sequence selected from the group of sequences consisting of SEQ ID NO 1, SEQ ID NO 2, and SEQ ID NO 3.

33. A method for preparing a functional molecule comprising:
identifying a functional biomolecule to be generated,
identifying the attributes within the functional biomolecule that are necessary for function,
identifying a precursor food source that contains a precursor molecule with the attributes necessary for function,
identifying an agent that can be used to release from the precursor molecule a compound comprising the attributes necessary for function, and
treating the precursor food source with the agent to release from the precursor molecule a molecule that contains the attributes necessary for function.

34. The method of claim 33 comprising purifying the functional biomolecule from a processed precursor food source.

35. The method of claim 33 comprising concentrating the functional biomolecule in a processed precursor food source.

36. The method of claim 33 comprising preparing an extract of a processed precursor food source that contains an elevated concentration of the biomolecule.

37. The method of claim 33 wherein the agent releases the biomolecule from the precursor molecule.

38. A food product comprising a digested precursor molecule and a biomolecule that is released from the precursor molecule during the digestion.

39. The bioactive agent of claim 27, wherein the food stuff is rice.


SEQUENCE LISTING (9 sequences)

SEQ ID NO: 1 - protein, 15 aa, Arabidopsis thaliana
YSCRYFGYLVSKKKY

SEQ ID NO: 2 - protein, 15 aa, Oryza sativa
HSCRYFGYLVSRKKY

SEQ ID NO: 3 - protein, 15 aa, Oryza sativa
SACRCFGYMVSKKKY

SEQ ID NO: 4 - protein, 15 aa, Oryza sativa
FDGVDFSEPLTRARF

SEQ ID NO: 5 - protein, 10 aa, Oryza sativa
VWEKPWMDFK

SEQ ID NO: 6 - protein, 6 aa, Oryza sativa
PWMDFK

SEQ ID NO: 7 - protein, 12 aa, Oryza sativa
PWMDFKELQEFK

SEQ ID NO: 8 - protein, 5 aa, Oryza sativa
PWMDF

SEQ ID NO: 9 - protein, 9 aa, Oryza sativa
VWEKPWMDF